SEQUENCE LISTING (4 sequences)

SEQ ID NO: 1 - protein, 4 aa, Artificial Sequence
  Synthetic construct
IPVE

SEQ ID NO: 2 - protein, 5 aa, Artificial Sequence
  Synthetic construct
IEGTL

SEQ ID NO: 3 - protein, 4 aa, Artificial Sequence
  Synthetic construct
IVDI

SEQ ID NO: 4 - protein, 9 aa, Artificial Sequence
  Synthetic construct
VAPEEHPTL

(19) United States
(12) Patent Application Publication
(10) Pub. No.: US 2023/0002457 A1
(43) Pub. Date: Jan. 5, 2023

(54) PEPTIDES FOR REGULATING GLUCOSE

(71) Applicants: Loïc Henaux, Repentigny (CA); André Marette, Québec City (CA); Laurent Bazinet, Québec City (CA); Cooke Aquaculture Inc., Saint John (CA); Université Laval, Québec City (CA)

(72) Inventors: Loïc Henaux, Repentigny (CA); André Marette, Québec City (CA); Laurent Bazinet, Québec City (CA)

(21) Appl. No.: 17/782,869

(22) PCT Filed: Dec. 4, 2020
(86) PCT No.: PCT/CA2020/051665

§ 371 Date: Jun. 6, 2022

Related U.S. Application Data
(60) Provisional application No. 62/944,794, filed on Dec. 6, 2019.

(51) Int. Cl. C07K 14/46; A61P 3/10; A61K 9/00

(52) U.S. Cl. CPC C07K 14/461; A61P 3/10; A61K 9/0053; A61K 9/0019

(57) ABSTRACT

Novel peptides and their uses are provided. In particular, the peptides are useful for increasing glucose uptake or decreasing hepatic glucose production. The peptides are also useful for regulating glucose levels and/or treating diabetes in a subject.

### CROSS-REFERENCE TO RELATED APPLICATION

This disclosure claims the benefit of U.S. provisional application No. 62/944,794, filed Dec. 6, 2019, the contents of which is incorporated herein by reference in its entirety.

### FIELD

This disclosure relates to novel glucoregulatory peptides and their use for increasing glucose uptake and decreasing hepatic glucose production. The disclosure also relates to use of the peptides for treating diabetes.

### BACKGROUND

Type 2 diabetes (T2D) is a complex multifactorial disorder resulting from insulin resistance in peripheral tissues such as skeletal muscle, and pancreatic β-cell dysfunction (Stumvol et al., 2005). According to a recent report from the International Diabetes Federation, in 2000, 151 million people aged between 18 to 99 years had T2D. In 2017, 425 million people were suffering from T2D (International Diabetes Federation, 2017). This disease is growing at a fast rate (Wild et al., 2004).

Salmon Protein Hydrolysate (SPH) has been tested in in vitro studies. SPHs may have effects on glucose uptake (Chevrier et al., 2015, Roblet et al., 2016) and hepatic glucose production (Chevrier et al., 2015). These bioactivities may be caused by the presence of low molecular (<1 kDa) bioactive peptides (BPs) in the SPHs (Chevrier et al., 2015, Roblet et al., 2016). Nevertheless, the identification of these BPs has never been done.

### SUMMARY

In this context, the inventors aimed to generate bioactive fractions useful for the treatment of T2D and to identify potential peptide sequences responsible for this bioactivity.

Provided herein are glucoregulatory peptides, compositions and combinations, and methods and uses thereof.

Accordingly an aspect of the present disclosure includes a peptide comprising (i) an amino acid sequence as shown in SEQ ID NO: 1 (IPVE); or (ii) a peptide comprising at least 50 or 75% sequence identity with the amino acid sequence as shown in SEQ ID NO: 1 that increases glucose uptake.

A further aspect includes a peptide comprising (i) an amino acid sequence as shown in any one of SEQ ID NO: 2 (IEGTL), SEQ ID NO: 3 (IVDI), or SEQ ID NO: 4 (VAPEEHPTL), or (ii) a peptide comprising at least 33, 40, 50, 67, 75, 80, or 90% sequence identity with the amino acid sequence as shown in any one of SEQ ID NOs: 2-4 that decreases hepatic glucose production.

In an embodiment, the peptide consists of the amino acid sequence of any one of SEQ ID NOs: 1-4.

In an embodiment, the peptide further comprises additional amino acids and is at least: 5, 6, 7, 8, 9, 10, 11, 12, 13, 14, 15, 16, 17, 18, 19, or 20 amino acids in length. In an embodiment, the peptide is less than 50, 45, 40, 35, 30, 25, 20, 15, 14, 13, 12, 11, 10, 9, 8, 7, 6 or 5 amino acids in length and comprises an amino acid sequence encoding a peptide that increases glucose uptake or decreases hepatic glucose production as described herein.

In an embodiment, the peptide is modified for cell permeability, stability or bioavailability.

Also provided is a composition comprising a peptide described herein and a carrier.

Further provided is a composition or combination comprising (i) at least two peptides described herein and optionally (ii) at least two, at least three or four peptides of any one of SEQ ID NOs: 1-4 and a carrier.

Yet a further aspect includes a method of increasing glucose uptake in a subject in need thereof, the method comprising administering to the subject a peptide, composition, or combination described herein.

Also provided is a method of decreasing hepatic glucose production in a subject in need thereof, the method comprising administering to the subject a peptide, composition, or combination described herein.

A further aspect includes a method of regulating glucose levels in a subject in need thereof, the method comprising administering to the subject a peptide, composition, or combination described herein.

Yet a further aspect includes a method of treating diabetes, optionally type 1 or type 2 diabetes, in a subject in need thereof, the method comprising administering to the subject a peptide, composition, or combination described herein.

In an embodiment, the subject is a diabetic subject.

In an embodiment, the subject is a mammal, optionally a dog, cat, horse, or human. In one embodiment, the subject is a human.

In an embodiment, the peptide, composition, or combination is administered or is for use orally or intravenously.

Also provided is a method of obtaining the peptides described herein, the method comprising:
providing a homogenized salmon frame or fraction;
precipitating proteins from the homogenized fraction;
hydrolyzing the precipitated proteins to form a hydrolyzed solution;
filtering the hydrolyzed solution using an ultrafiltration membrane to generate a filtrate; and
isolating the peptides from the filtrate.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### DRAWINGS

Embodiments are described below in relation to the drawings in which:

FIG. 1 shows a schematic EDUF cell in a) configuration 1 for the generation of CFFC₂ from the fractionation of C_{FFC} (Cationic final feed compartment) and b) configuration 2 for the generation of and AFFC₂ from the fractionation of A_{FFC} (Anionic final feed compartment). The C_{FFC} and A_{FFC} fractions were generated from previous work, Henaux et al, 2019.

FIG. 2 shows evolution of peptide concentration in anionic (K_{CL}−) and cationic (K_{CL}+) peptide recovery compartments.

FIG. 3 shows the UV spectra of the recovery compartments after 4 h of EDUF separation: a) the chromatogram of A_{FFC}, A_{FFC2} and K_{CL+} separated in parts I and II, and b) the chromatogram of C_{FFC}, C_{FFC2} and K_{CL-} separated in parts I, II and III.

FIG. 4 shows effects of synthetic peptides on the glucose uptake modulation in L6 skeletal muscle cells in a) basal and b) insulin-stimulated conditions. An asterisk indicates that mean values are significantly different (P<0.05) from the control's mean value.

FIG. 5 shows the dose-response effect of IPVE on the glucose uptake modulation in L6 skeletal muscle cells in a) basal or b) insulin stimulated conditions. An asterisk indicates that mean values are significantly different (P<0.05) from the control's mean value.

FIG. 6 shows effects of synthetic peptides on in vitro hepatic production from FAO cells in a) basal and b) insulin stimulated conditions. An asterisk indicates that mean values are significantly different (P<0.05) from the control's mean value.

### DESCRIPTION OF VARIOUS EMBODIMENTS

Unless otherwise indicated, the definitions and embodiments described in this and other sections are intended to be applicable to all embodiments and aspects of the present disclosure herein described for which they are suitable as would be understood by a person skilled in the art.

Unless otherwise defined, scientific and technical terms used in connection with the present disclosure shall have the meanings that are commonly understood by those of ordinary skill in the art.

Terms of degree such as “about”, “substantially”, and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. All ranges disclosed herein are inclusive of the endpoints, and the endpoints are independently combinable with each other.

A “therapeutically effective amount” is intended to mean that amount of a compound that is sufficient to treat, prevent or inhibit a disease or condition such as T2D and/or hyperglycemia. The amount of a given compound of the present disclosure that will correspond to such an amount will vary depending upon various factors, such as the given compound, the composition, the route of administration, the type of disease or disorder, the identity of the subject or host being treated, and the like, but can nevertheless be routinely determined by one skilled in the art. In one embodiment, a “therapeutically effective amount” is an amount sufficient to have a desired effect on a subject, such as reducing hyperglycemia, increasing cellular glucose uptake and/or decreasing hepatic glucose production.

### Compositions of Matter:

### Peptides, Nucleic Acids, Vectors and Recombinant Cells

The disclosure provides peptides that have effects, such as to increase glucose uptake or decrease hepatic glucose production. The peptides described herein can increase glucose uptake or decrease hepatic glucose production in vitro or in vivo.

Glucose uptake can typically occur in one of two ways: passively (such as by facilitated diffusion) or actively (such as by secondary active transport).

An increase in glucose uptake by a cell refers to the increase in the amount, whether active or passive, of glucose that is taken up by the cell. Thus, reducing glucose uptake of a cell includes the reduction of uptake of glucose by the cell from the extracellular environment, e.g., from blood vessels or surrounding environment. Reducing glucose uptake includes a reduction or decrease in the uptake of glucose by at least some cells of a subject. The terms higher or increase refer to any increase above normal homeostatic levels. For example, control levels are in vitro, ex vivo, or in vivo levels prior to, or in the absence of, addition of an agent. Thus, the increase can be at least: 10, 20, 30, 40, 50, 60, 70, 80, 90, 100%, or any amount of increase in between as compared to native or control levels.

Peptides provided by the present disclosure are set out in Table 1 (SEQ ID NOs: 1-4).

As used herein, the term “peptide” refers to two or more amino acids linked by a peptide bond, and includes synthetic and natural peptides as well as peptides that are modified. Various lengths of peptides are contemplated herein.

The peptide can for example be 4-50 amino acids in length as amino acids may be added to the peptides in Table 1, optionally 7-30 amino acids in length or at least 25 or 30 amino acids in length. The peptide can for example be any number of amino acids between 4 and 30.

Accordingly, in one embodiment, the peptide comprises an amino acid sequence as shown in any one of SEQ ID NOs: 1-4, or a conservatively substituted variant thereof.

Also provided is a peptide that is a part of a sequence described herein, optionally a part of any one of SEQ ID NOs: 1-4, that retains all or part of the biological activity.

The term “part” with reference to amino acids over 4 amino acids long means at least 4 contiguous amino acids of the reference sequence. The reference sequence can for example by any one of SEQ ID NOs: 1-4, or a conservatively substituted variant thereof.

In another embodiment, the peptide consists essentially of, or consists of an amino acid sequence as shown in any one of SEQ ID NOs: 1-4, or a conservatively substituted variant thereof.

In another embodiment, the peptide comprises an amino acid sequence with at least: 25, 30, 33, 40, 50, 60, 67, 70, 75, 80, 90, 95 or 99% sequence identity with the amino acid sequence as shown in any one of SEQ ID NOs: 1-4 or a part thereof. In another embodiment, the peptide comprises or consists of an amino acid sequence comprising at least 4, 5, 6, 7 or 8 contiguous amino acids of SEQ ID NOs: 1-4.

In particular, described herein is the peptide “IPVE” comprising the amino acid sequence set out in SEQ ID NO: 1, or a conservatively substituted variant thereof, wherein the peptide increases glucose uptake.

Also described herein is the peptide “IEGTL” comprising the amino acid sequence set out in SEQ ID NO: 2, or a conservatively substituted variant thereof, wherein the peptide decreases hepatic glucose production.

Also described herein is the peptide “IVDI” comprising the amino acid sequence set out in SEQ ID NO: 3, or a conservatively substituted variant thereof, wherein the peptide decreases hepatic glucose production.

Also described herein is the peptide “VAPEEHPTL” comprising the amino acid sequence set out in SEQ ID NO: 4, or a conservatively substituted variant thereof, wherein the peptide decreases hepatic glucose production.

The peptide comprising any one of SEQ ID NOs: 1-4 may further comprise additional amino acids and be at least: 5, 6, 7, 8, 9, 10, 11, 12, 13, 14, 15, 16, 17, 18, 19, or 20 amino acids in length. In an embodiment, the peptide is less than 50, 45, 40, 35, 30, 25, 20, 15, 14, 13, 12, 11, 10, 9, 8, 7, 6 or 5 amino acids in length and comprises an amino acid sequence encoding a peptide that increases glucose uptake or decreases hepatic glucose production as described herein, such as any one of SEQ ID NOs: 1-4.

In one embodiment, the disclosure provides a peptide that has at least: 25, 30, 33, 40, 50, 60, 67, 70, 75, 80, 90, 95 or 99% sequence identity with any one of SEQ ID NOs: 1-4.

Sequence identity can be calculated according to methods known in the art. Sequence identity is optionally assessed by the algorithm of BLAST version 2.1 advanced search. BLAST is a series of programs that are available, for example, online from the National Institutes of Health. The advanced blast search is set to default parameters. (ie Matrix BLOSUM62; Gap existence cost 11; Per residue gap cost 1; Lambda ratio 0.85 default). References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_141; Altschul, S. F., Madden, T. L., Schiffer, A. A., Zhang, J., Zhang, Z., Miller, W. & Lipman, D. J. (1997) “Gapped BLAST and PSI_BLAST: a new generation of protein database search programs.” Nucleic Acids Res. 25:33893402; Zhang, J. & Madden, T. L. (1997) “PowerBLAST: A new network BLAST application for interactive or automated sequence analysis and annotation.” Genome Res. 7:649656. In addition, percent identity between two sequences may be determined by comparing a position in the first sequence with a corresponding position in the second sequence. When the compared positions are occupied by the same nucleotide or amino acid, as the case may be, the two sequences are conserved at that position. The degree of conservation between two sequences is often expressed, as it is here, as a percentage representing the ratio of the number of matching positions in the two sequences to the total number of positions compared.

As used herein, the term “conservatively substituted variant” refers to a variant with at least one conservative amino acid substitution. A “conservative amino acid substitution” as used herein, refers to the substitution of an amino acid with similar hydrophobicity, polarity, and R-chain length for one another. In a conservative amino acid substitution, one amino acid residue is replaced with another amino acid residue without abolishing the protein's desired properties. Without the intention of being limited thereby, in one embodiment, the substitutions of amino acids are made that preserve the structure responsible for the ability of the peptide to increase glucose uptake or decrease hepatic glucose production as disclosed herein. Examples of conservative amino acid substitutions include:

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

In one embodiment, the peptides described herein are optionally modified for cell permeability, improved stability, and/or better bioavailability. These modifications include, without limitation, peptide conjugation, peptide cyclization, peptide end modification (e.g. N-acetylation or C-amidation, side chain modifications including the incorporation of non-coded amino acids or non-natural amino acids, N-amide nitrogen alkylation, chirality changes (incorporation of or replacement of L-amino acids with D-amino acids), generation of pseudopeptides (e.g. amide bond surrogates), or peptoids, or azapeptides or azatides). In one embodiment, the peptides described herein are modified by the addition of a lipophilic moiety.

The peptides described above may be prepared using recombinant DNA methods. These peptides may be purified and/or isolated to various degrees using techniques known in the art. Accordingly, nucleic acid molecules having a sequence which encodes a peptide of the disclosure may be incorporated according to procedures known in the art into an appropriate expression vector which ensures good expression of the protein. Possible expression vectors include but are not limited to cosmids, plasmids, or modified viruses (e.g., replication defective retroviruses, adenoviruses and adeno-associated viruses), so long as the vector is compatible with the host cell used. The expression “vectors suitable for transformation of a host cell”, means that the expression vectors contain a nucleic acid molecule encoding a peptide of the disclosure and regulatory sequences, selected on the basis of the host cells to be used for expression, which are operatively linked to the nucleic acid molecule. “Operatively linked” is intended to mean that the nucleic acid is linked to regulatory sequences in a manner which allows expression of the nucleic acid.

The peptides may be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

In one embodiment, the peptides may be modified with a detectable label. For example, in one embodiment the peptide is fluorescently, radioactively or immunologically labeled.

The peptides may also be modified with an enhancer moiety. Accordingly, another aspect provides a compound comprising a peptide described herein and an enhancer moiety. In one embodiment, the peptide is conjugated directly or indirectly to the enhancer moiety. As used herein, an enhancer moiety can increase or enhance the activity of the peptide. For example, the enhancer may be a permeability enhancer, a stability enhancer or a bioavailability enhancer. The enhancer moiety is optionally selected from a protein carrier, or a polymer carrier. In one embodiment, the enhancer moiety is a carrier protein, thereby forming a fusion protein. In another embodiment, the enhancer moiety is a PEG moiety.

The peptides may also be modified with a cell-penetrating moiety. As used herein, the term “cell-penetrating moiety” refers to a moiety that promotes cellular uptake of the peptide upon delivery to a target cell. Examples of cell-penetrating moieties include cell-penetrating peptides that translocate across the plasma membrane of eukaryotic cells at higher levels than passive diffusion. In one embodiment, the cell-penetrating peptide can translocate the nuclear membrane of a cell to enter the nucleus. In another embodiment, the cell-penetrating peptide can enter the nucleolus.

In one embodiment, the cell-penetrating peptide is an amphipathic peptide comprising both a hydrophilic (polar) domain and a hydrophobic (non-polar) domain. Cell-penetrating peptides can include sequences from membrane-interacting proteins such as signal peptides, transmembrane domains and antimicrobial peptides.

The peptides described herein can also be conjugated to a carrier protein, thereby forming a fusion protein.

The disclosure also includes nucleic acids that encode the peptides described herein. As used herein, the term “nucleic acids” includes isolated nucleic acids. In one embodiment, the disclosure provides nucleic acids that encode a peptide comprising or consisting of any one of SEQ ID NOs: 1-4 or any peptide described herein.

In another embodiment the disclosure provides a nucleic acid having at least 50, 60, 67, 70, 80, 90, 95 or 99% sequence identity with a nucleic acid that encodes a peptide comprising or consisting of any one of SEQ ID NOs: 1-4, a nucleic acid that hybridizes to a nucleic acid that encodes a peptide comprising or consisting of any one of SEQ ID NOs: 1-4 or any peptide described herein under at least moderately stringent hybridization or stringent hybridization conditions.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 (Na+))+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation) −5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in Ausubel, 1989 and in Sambrook et al., 1989.

The disclosure further contemplates a vector comprising a nucleic acid described herein, optionally a recombinant expression vector containing a nucleic acid molecule that encodes a peptide of the disclosure and the necessary regulatory sequences for the transcription and translation of the inserted protein-sequence. In an embodiment, the vector is a viral vector such as a retroviral, lentiviral, adenoviral or adeno-associated viral vector.

Recombinant expression vectors can be introduced into host cells to produce a transformed host cell for the purpose of producing the peptides described herein. The term “transformed host cell” is intended to include prokaryotic and eukaryotic cells which have been transformed or transfected with a recombinant expression vector of the disclosure. The terms “transformed with”, “transfected with”, “transformation” and “transfection” are intended to encompass introduction of nucleic acid (e.g. a vector) into a cell by one of many possible techniques known in the art. Suitable host cells include a wide variety of prokaryotic and eukaryotic host cells.

Also provided in another aspect is a recombinant cell expressing a peptide, nucleic acid, vector or compound described herein. In an embodiment, the cell is a bacterial cell, yeast cell, a mammalian cell, or a plant cell.

### Compositions and Combinations of Peptides

The disclosure also provides a composition comprising one or more of the peptides described herein. Also provided is a combination of two or more peptides described herein.

In one aspect, the composition comprises a peptide described herein and a carrier. In another embodiment, the composition or combination comprises at least two peptides described herein, optionally at least two, at least three or at least four peptides of SEQ ID NOs: 1-4 and a carrier.

In one embodiment, the carrier is a carrier acceptable for administration to humans.

As used herein, the term “acceptable carrier” is intended to include any and all solvents, dispersion media, coatings, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Optional examples of such carriers or diluents include, but are not limited to, water, saline, ringer's solutions and dextrose solution.

In one embodiment, a composition or combination described herein is formulated to be compatible with its intended route of administration. Examples of routes of administration include oral and parenteral, e.g. intravenous, intradermal, subcutaneous.

For example, in one embodiment, the active ingredient such as a peptide described herein is prepared with a carrier that will protect it against rapid elimination from the body, such as a sustained/controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

In one embodiment, oral or parenteral compositions or combinations are formulated in dosage unit form for ease of administration and uniformity of dosage. Dosage unit form as used herein refers to physically discrete units suited as unitary dosages for the subject to be treated; each unit containing a predetermined quantity of active ingredient calculated to produce the desired therapeutic effect in association with the required carrier. The specification for the dosage unit forms are dictated by and directly dependent on the unique characteristics of the active ingredient and the particular therapeutic effect to be achieved, and the limitations inherent in the art of preparing such an active ingredient for the treatment of individuals.

In one embodiment, the compositions described herein comprise an agent that enhances its function, such as, for example, insulin, other diabetes medication(s), omega 3, and/or polyphenols. The composition can also contain other active ingredients as necessary or beneficial for the particular indication being treated, optionally those with complementary activities that do not adversely affect each other. Such active ingredients are suitably present in combination in amounts that are effective for the purpose intended.

### Methods and Uses

The disclosure also provides uses and methods relating to the peptides, compositions, and combinations described herein.

Some of the peptides disclosed herein increase glucose uptake by cells, while others decrease hepatic glucose production. Accordingly, the peptides, compositions, and combinations of the present disclosure are useful for regulating blood glucose levels in a subject and optionally for treating diabetes in a subject. In one embodiment, the peptides described herein are useful for reducing hyperglycemia in a subject, optionally in a subject with T2D.

In one embodiment, the methods and uses include the administration to a subject or use in a subject of a peptide, composition or combination as described herein. In one embodiment, the subject is a diabetic subject. In one embodiment, the subject is a mammal, optionally a dog, cat, horse, or human. In one embodiment, the mammal is a human. In one embodiment, the peptide, composition, or combination is administered orally or intravenously. In another embodiment, the peptide, composition, or combination is for use orally or intravenously.

### Methods and Uses of Increasing Glucose Uptake:

The disclosure provides a method of increasing glucose uptake in a subject in need thereof, the method comprising administering to the subject a peptide, composition, or combination described herein. Also provided is use of a peptide, composition, or combination disclosed herein to increase glucose uptake. In another embodiment, a peptide, composition, or combination disclosed herein is used in the manufacture of a medicament to increase glucose uptake. In yet another embodiment, a peptide, composition, or combination disclosed herein is for use in treating hyperglycemia.

As used herein, the term “hyperglycemia” refers to higher than normal fasting blood glucose concentration, optionally at least 125 mg/dL.

### Methods and Uses of Decreasing Hepatic Glucose Production:

The disclosure further provides a method of decreasing hepatic glucose production in a subject in need thereof, the method comprising administering to the subject a peptide, composition, or combination described herein. Also provided is use of a peptide, composition, or combination disclosed herein to decrease hepatic glucose production. In another embodiment, a peptide, composition, or combination disclosed herein is used in the manufacture of a medicament to decrease hepatic glucose production. In yet another embodiment, a peptide, composition, or combination disclosed herein is for use in treating hepatic hyperglycemia.

### Methods and Uses of Regulating Glucose Levels:

The disclosure further provides a method of regulating glucose levels in a subject in need thereof, the method comprising administering to the subject a peptide, composition, or combination described herein. Also provided is use of a peptide, composition, or combination disclosed herein to regulate glucose levels. In another embodiment, a peptide, composition, or combination disclosed herein is used in the manufacture of a medicament to regulate glucose levels. In yet another embodiment, a peptide, composition, or combination disclosed herein is for use in regulating glucose levels.

Regulating glucose levels comprises the lowering of hyperglycemic glucose levels to a normoglycemic range. Optionally a normoglycemic range is 70-130 mg/dL. Optionally the glucose levels are maintained substantially in that normoglycemic, for example for at least: 30, 60, 90, 120, 180 or 240 minutes. For example, 30-60, 30-120, or 30-240 minutes.

### Methods and Uses of Treating Prediabetes:

The disclosure further provides a method of treating prediabetes in a subject in need thereof, the method comprising administering to the subject a peptide, composition, or combination described herein. Also provided is use of a peptide, composition, or combination disclosed herein to treat prediabetes. In another embodiment, a peptide, composition, or combination disclosed herein is used in the manufacture of a medicament to treat prediabetes. In yet another embodiment, a peptide, composition, or combination disclosed herein is for use in treating prediabetes.

Prediabetes is also referred to as “impaired glucose tolerance” or “impaired fasting glucose” and refers to blood glucose levels that are higher than a normal fasting blood glucose concentration, but are not high enough to be classified as type-2 diabetes. For example, from 100 to 125 mg/dL.

### Methods and Uses of Treating Diabetes:

The disclosure further provides a method of treating diabetes, optionally type 1 or type 2 diabetes, in a subject in need thereof, the method comprising administering to the subject a peptide, composition, or combination described herein. Also provided is use of a peptide, composition, or combination disclosed herein to treat diabetes, optionally type 1 or type 2 diabetes. In another embodiment, a peptide, composition, or combination disclosed herein is used in the manufacture of a medicament to treat diabetes, optionally type 1 or type 2 diabetes. In yet another embodiment, a peptide, composition, or combination disclosed herein is for use in treating diabetes, optionally type 1 or type 2 diabetes.

### Methods and Uses of Treating Metabolic Syndrome:

The disclosure provides a method of treating metabolic syndrome in a subject in need thereof by reducing one or more of hyperglycemia and hypertension, the method comprising administering to the subject a peptide, composition, or combination described herein. Also provided is use of a peptide, composition, or combination disclosed herein to treat metabolic syndrome. In another embodiment, a peptide, composition, or combination disclosed herein is used in the manufacture of a medicament to treat metabolic syndrome. In yet another embodiment, a peptide, composition, or combination disclosed herein is for use in treating metabolic syndrome.

### Methods of Obtaining Peptides:

The disclosure further provides a method of obtaining the peptides disclosed herein. In one embodiment the method comprises providing a homogenized salmon frame or fraction, precipitating proteins from the homogenized fraction, hydrolyzing the precipitated proteins to form a hydrolyzed solution, filtering the hydrolyzed solution using an ultrafiltration membrane to generate a filtrate, and isolating the peptides from the filtrate, optionally isolating peptides of SEQ ID NO: 1, SEQ ID NO: 2, SEQ ID NO: 3, and SEQ ID NO: 4 into separate fractions.

In an embodiment, precipitating the proteins is performed by isoelectric precipitation at pH 4.5.

Hydrolysis of precipitated proteins may be carried out with a variety of enzymes known to a person skilled in the art.

In an embodiment, hydrolyzing the peptides precipitated proteins is performed using trypsin, chymotrypsin, pepsin, or any combination thereof.

Ultrafiltration may comprise several techniques known to a skilled person. In an embodiment, ultrafiltration comprises-pressure driven ultrafiltration. In another embodiment ultrafiltration comprises electrodialysis with an ultrafiltration membrane.

Ultrafiltration membranes comprise pores that may be, for example, 0.1 to 0.001 μm.

In an embodiment, the ultrafiltration membrane has a molecular weight cutoff of 1 kDa.

Peptide isolation may be performed using a variety of methods known to a skilled person and may include various chromatography methods such as size-exclusion, affinity purification, and ion exchange.

In an embodiment, isolating the peptides is performed using reverse-phase liquid chromatography.

Also provided is a method of producing a peptide as described herein comprising culturing a host cell that expresses a nucleic acid encoding the peptide, such as a peptide selected from SEQ ID NO: 1-4, and optionally isolating the peptide.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1—Production of Salmon Fractions to Obtain Isolated Peptide

### Materials and Electrodialysis Cell

### Hydrolyzate Preparation:

Salmon Protein Hydrolysate (SPH) was produced according to the procedure described previously by Chevrier et al, (2015). Briefly, salmon frames were thawed, mechanically deboned and homogenized in a 1.0 M NaOH solution. Then, fish proteins were isoelectrically precipitated, recovered and a sequential hydrolysis was carried out with pepsin, then trypsin and chymotrypsin. Once hydrolysis was complete, the supernatant was filtered through a 5 μm pore size paper filter to remove any insoluble fat or protein. Finally, the filtrate was subsequently ultrafiltered using a Prep/Scale Tangential Flow Filtration (TFF) 2.5 ft² cartridge with 1 kDa exclusion limit (Millipore Corporation, Bedford, Mass., USA). Permeates containing peptides with molecular weights <1 kDa were collected, demineralized by electrodialysis and freeze-dried.

### Membranes:

One ultrafiltration membrane made of polyether sulfone (PES) with a molecular weight exclusion limit of 50 kDa, was purchased from Synder filtration (Vacaville, Calif., USA). Food grade Neosepta™ CMX-SB cationic membranes and Neosepta AMX-SB anionic membranes were obtained from Astom (Tokyo, Japan).

### Electrodialysis Configurations:

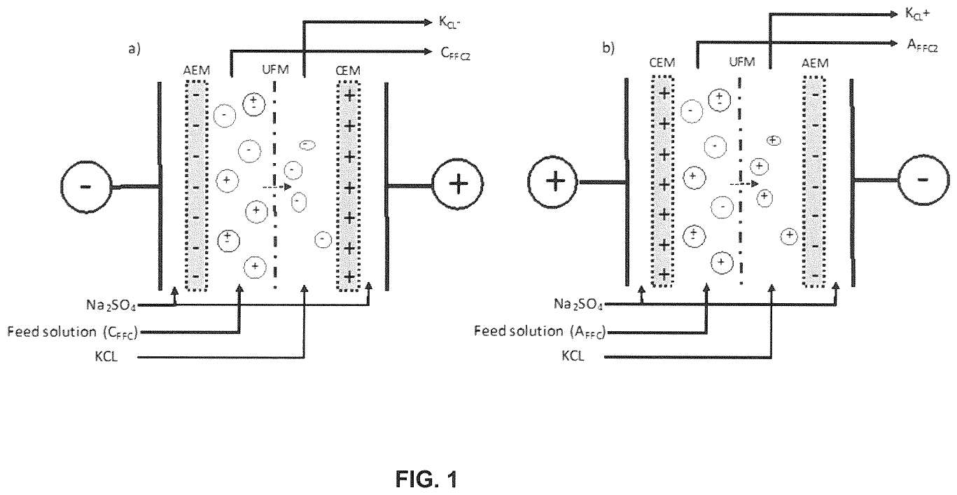

The electrodialysis cell used for the experiment was an MP type cell with an effective surface area of 100 cm², manufactured by ElectroCell Systems AB Company (Täby, Sweden). The cell was composed of one anion-exchange membrane (AEM), one cation-exchange membranes (CEM), one ultrafiltration membrane (UFMs) with MWCO 50 kDa as illustrated in FIG. 1. The electrodes used were a dimensionally-stable anode (DSA) and a 316 stainless steel cathode. The electrical potential for the Electrodialysis with Ultrafiltration Membrane (EDUF instead of EDFM since the filtration membrane was an UF membrane) was supplied by a variable 0-100 V power source. Two different cell configurations allowing the separation of cationic or anionic charged peptides from salmon protein hydrolysate were tested in this study. In both configurations. The solutions were circulated using three centrifugal pumps and the flow rates were set at 2 L/min using flow meters (the electrode rinsing solution was maintained at 4 L/min and split in half between the anode and the cathode compartments) (Blue-White Industries Ltd. Huntington Beach, Calif., USA).

First configuration: The first EDUF cell configuration, shown in FIG. 1a was arranged for the separation of anionic peptides. The cell was divided into three closed loops; one contained 1.5 L of a KCl solution (2 g/L) for the recovery and concentration of anionic peptides (K_{CL}−). The feed solution consisting of the C_{ationic Final Feed Compartment} (C_{FFC}) generated from a previous EDUF separation (Henaux et al. 2019) was circulated in the compartment between the UFM and CEM. The recovery solution from the feed compartment was called C_{FFC2}. The last loop contains the electrode rinsing solution (20 g/L, Na₂SO₄, 3 L). Which was split into two streams circulating into both electrolyte compartments.

Second configuration: In a second configuration (FIG. 1b), the compartment containing a KCl solution circulating between the UFM and CEM allowed the recuperation of cationic peptides (K_{CL}+). The feed solution was circulated in the compartment between the UFM and AEM. The feed solution consisting of the A_{nionic Final Feed Compartment} (A_{FFC}) generated from a previous EDUF separation (Henaux et al. 2019), and the final solution recovered in this compartment was called A_{FFC2}. The rinsing electrode solution was circulated into both electrode compartments as for the anionic configuration.

### Electroseparation Protocol

The spray dried SPH was diluted with demineralized water at a final protein concentration of 0.7% (w/v) and the EDUF fractionation was performed for 4 h. EDUF experiments were performed in batches for both cell configurations using constant electrical field strength of 6 V/cm (corresponding to a current density varying between 0.005 and 0.008 A/cm² during the treatment). The system was run at controlled temperature (˜16° C.) to prevent growth of microorganisms (Suwal, Roblet, Amiot, & Bazinet, 2015). The pH of SPH and recovery (KCl) solutions were adjusted to pH 6 before each run with 0.1 N NaOH and/or 0.1N HCl and maintained constant thereafter (Roblet et al., 2016). For each treatment 10 mL-sample of SPH and recovery solutions were collected every hour before applying voltage and during the treatment to determine the peptide migration rate and their kinetics of migration. The electrical conductivity of the SPH feedstock and recovery solutions was maintained at a constant level by adding KCl, following the recommendations of Suwal et al, (2015) (Suwal et al., 2015). The current intensity, electrical potential differences of the AEM, CEM and UFMs were recorded every 30 min during EDUF treatment for both configurations. Finally, 3 replicates of each condition were performed. At the end of each replicate, a cleaning-in-place was performed according to the membrane manufacturer's instructions and the cell was dismantled before being reassembled.

### Analyses

### Evolution of Peptide Migration:

The peptide concentration in recovered compartments of both configurations, during and after 4 h of EDUF separation were determined using micro bicinchoninic acid (μBCA) protein assay reagents (Pierce, Rockford, Ill., USA). Assays were conducted on microplates by mixing 150 μL of the sample with 150 μL of the working reagent followed by incubation at 37° C. during 2 h. The microplate was then cooled to room temperature and the absorbance was read at 562 nm on a microplate reader (Thermomax, Molecular devices, Sunnyvale, Calif.). Concentration was determined with a standard curve of bovine serum albumin (BSA) following the manufacturer's indications.

### RP-UPLC and Mass Spectrometry Analyses:

RP-UPLC analyses were performed using a 1290 Infinity™ II UPLC (Agilent Technologies, Santa Clara, Calif., USA). The equipment consisted of a binary pump (G7120A), a multisampler (G7167B), an in-line degasser and a variable wavelength detector (VWD G7114B) adjusted to 214 nm. Peptides were diluted to 0.5 mg/mL and filtered through 0.22 μm PVDF filter into a glass vial. The sample was loaded (5 μL) onto an Acquity UPLC CSH 130 1.7 μm C18 column (2.1 mm i.d.×150 mm) (Waters Corporation, Milford, Mass., USA). The column was operated at a flow rate of 400 μL/min at 45° C. A linear gradient consisting of solvent A (LC-MS grade water with 0.1% formic acid) and solvent B (LC-MS grade ACN with 0.1% formic acid) was applied with solvent B going from 2% to 25% in 50 min holding until 53 min, after, ramping to 90% and holding until 57 min, then back to initial conditions. Each sample was run in triplicate for statistical evaluation of technical reproducibility.

A hybrid ion mobility quadrupole TOF mass spectrometer (6560 high definition mass spectrometry (IM-Q-TOF), Agilent, Santa Clara, USA) was used to identify and quantify the relative abundances of the peptides. All LC-MS/MS experiments were acquired using Q-TOF. Signals were recorded in positive mode at Extended Dynamic Range, 2 Ghz, 3200 m/z with a scan range between 100-3200 m/z. Nitrogen was used as the drying gas at 13.0 L/min and 150° C., and as nebulizer gas at 30 psig. The capillary voltage was set at 3500 V. The nozzle voltage was set at 300 V and the fragmentor at 400 V. The instrument was calibrated using an ESI-L low concentration tuning mix (G1969-85000, Agilent Technologies, Santa Clara, Calif., USA). Data acquisition and analysis were done using the Agilent Mass Hunter™ Software package (LC/MS Data Acquisition, Version B.07.00 and Qualitative Analysis for IM-MS, Version B.07.00 with BioConfirm Software). Additional search was done using the Spectrum Mill MS Proteomics Workbench Rev B.05.00.180.

### Statistical Analyses:

Evolutions of peptide concentration and relative abundance were subjected to a one way analysis of variance (ANOVA) using SAS software version 9.1 (SAS institute Inc., Cary, N.C., USA) with Tukey's post hoc tests at a significant P values of 0.05 for acceptance. In vitro glucose uptake assays were subjected to a one way ANOVA using SAS software version 9.1 (SAS institute Inc., Cary, N.C., USA) with Dunnett's post hoc test at a significant P values of 0.05 for acceptance. The relative energy consumption was compared by Student's t-test (P<0.05 as probability level for acceptance).

### Results and Discussion

### Evolution of Peptide Concentration and Final Migration Rates:

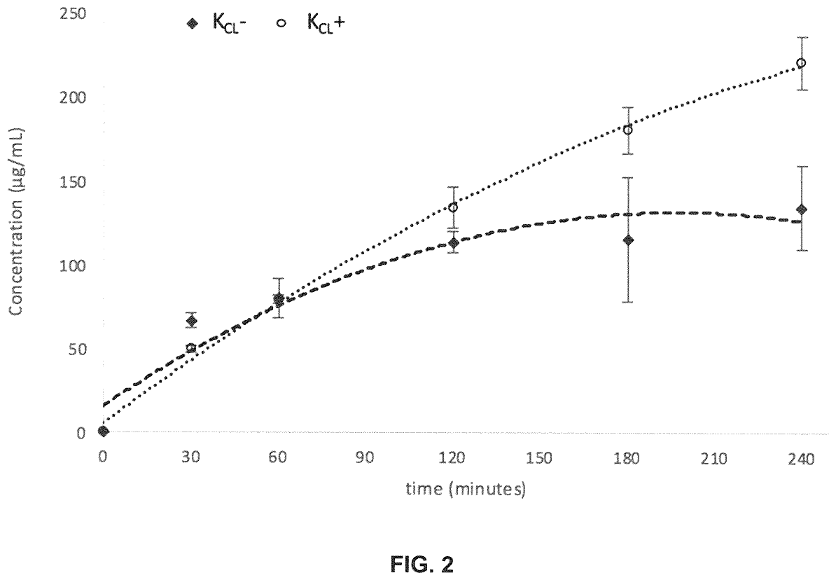

The evolution of peptide separation and concentration as a function of time in KCL compartments of both cationic and anionic EDUF configurations measured by micro-BCA method is represented in FIG. 2. Results demonstrated a higher migration of cationic peptides comparatively to the anionic peptide (P=0.007). Indeed, the final concentration obtained after 4 h of EDUF separation were 134.20±25.01 and 220.58±15.75 μg/mL for K_{CL}− and K_{CL}+, respectively. These results are in accordance with our previous works on the separation of salmon protein hydrolysate by EDUF (Henaux et al, 2019). Indeed, higher migration rates were obtained for cationic peptides. Without wishing to be bound by theory, two phenomena could explain these results. First, a higher concentration of cationic peptides in the USPH allowed a higher migration of these peptides in the recovery compartments (Henaux et al. 2019). Secondly, the migration through the ultrafiltration membrane was based on the peptide electrophoretic mobility (depending on the peptide charges and molecular weight). Due to a medium/low charge under the mass ratio resulting in a lower electrophoretic mobility the migration of anionic peptide towards the recovery compartments could be limited, and a higher voltage should be necessary to increase their migration (Aider, Arul, Mateescu, Brunet, & Bazinet, 2006). Indeed, previous work on the impact of field strength on chitosan oligomer migration have demonstrated that an increase of electric field strength allowed a higher migration of di-, tri- and tetramer (Aider, Brunet, & Bazinet, 2009). Moreover, from FIG. 2 it appeared that the migration of anionic peptide reached a plateau at 150 minutes while the migration of cationic peptides continued to increase linearly even after 240 minutes of EDUF experiments.

### Evolution of Peptide Profile During the EDUF Separation:

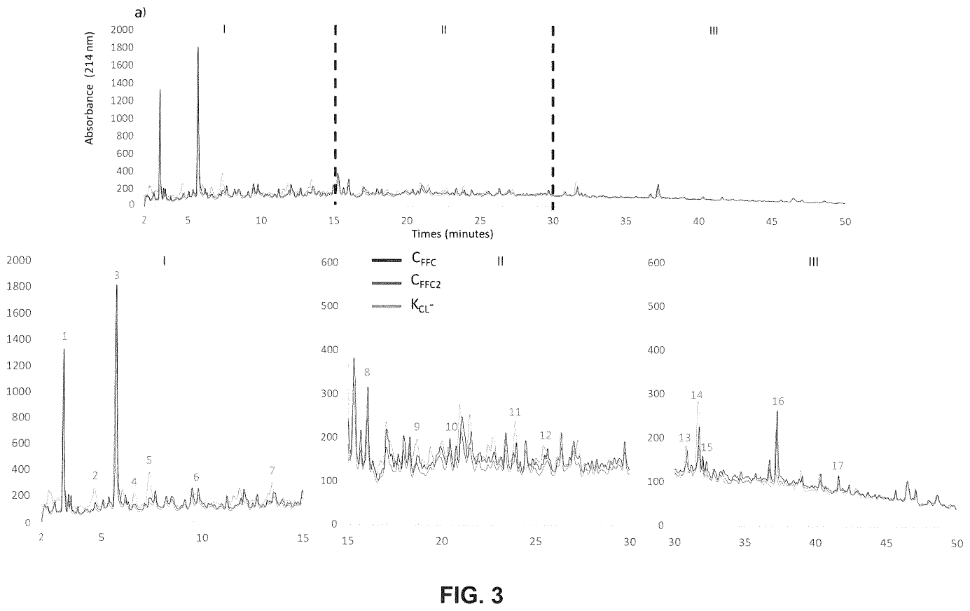

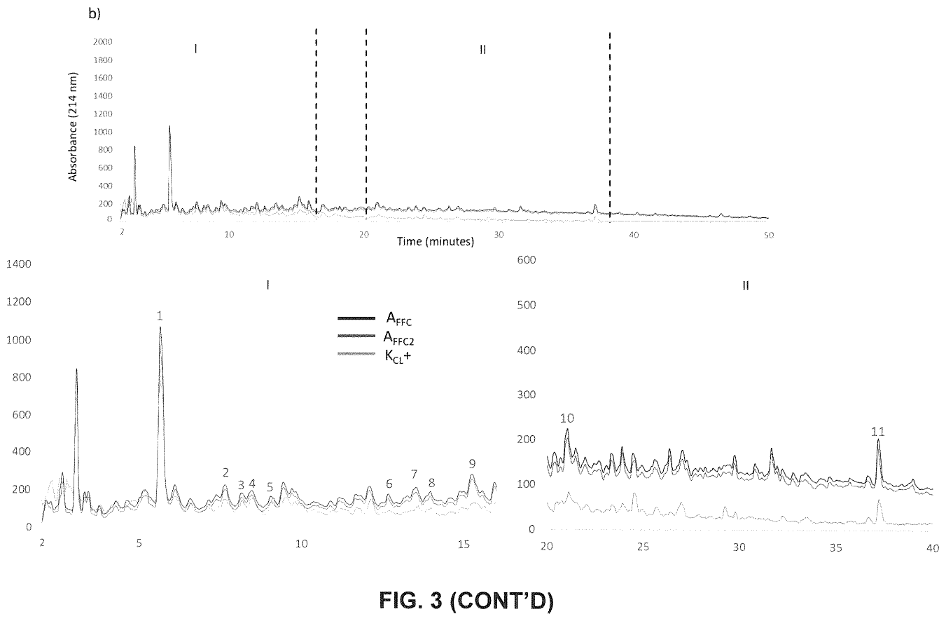

FIG. 3 represents the UV spectra of the recovery compartments after 4 h of EDUF separation. The chromatogram of A_{FFC}, A_{FFC2} and K_{CL}+ are presented in FIG. 3a and separated in two parts (parts I and II) while the chromatogram of C_{FFC}, C_{FFC2} and K_{CL}− are presented in FIG. 3b and separated in three parts (parts I, II and III).

As shown in FIG. 3a, no significant differences were observed between C_{FFC} and C_{FFC2}, both recovered in the same compartments after 4 h of EDUF separation. Nevertheless, significant differences (P<0.05) in the absorbance were observed between K_{CL}− and C_{FFC} and/or C_{FFC2}. Indeed, out of fifteen peaks, nine demonstrated an increase of their abundances in K_{CL}− (peak 1, 2, 3, 4, 5, 7, 9, 11 and 13), while six showed a decrease of their abundances in the K_{CL}− (peaks 6, 8, 10, 12, 14 and 15), comparatively to the C_{FFC} and/or the C_{FFC2}. Concerning configuration 2, significant differences were observed after the EDUF separation, between the A_{FFC} and the A_{FFC2} and/or the K_{CL}+. Most of the peaks (9 peaks) were decreased in the A_{FFC2} and the K_{CL}+ comparatively to the A_{FFC}, and only two peaks (peaks 5 and 6) demonstrated a significant increase of its abundance in the K_{CL}+ comparatively to the A_{FFC}.

Without being bound by theory, the differences in abundance observed between both compartments (feed and recovery compartments) may have been principally due to the selectivity of the EDUF process. Inventors demonstrated significant differences in terms of abundances and total peaks between the feed and recovery compartments after 360 minutes of EDUF separation. Moreover, a number of compounds were not able to cross the ultrafiltration membrane (those marked with one asterisk) while, some compounds were concentrated in the recovery compartments (those marked with two asterisks). For configuration 1, forty compounds were concentrated in K_{CL}. According to the potential anti-diabetic peptides previously identified (Henaux et al. 2019), three of these peptides were concentrated in the K_{CL-}. For configuration 2, three compounds were recovered in peak 5 and thirteen in peak 6, among which two compounds (743.36 and 724.36 Da) were observed only in K_{CL}+. For peak 5, three compounds were identified (598.37, 587.32 and 491.24 Da) but none were only found in the K_{CL}+. Nevertheless, a compound (587.32 Da) was found only in the feed compartment (in both A_{FFC} and A_{FFC2} fractions). Therefore, the increase in peak 5 area for K_{CL}+ was due to the concentration by EDUF of compounds 598.37 and/or 491.24 Da.

### Example 2— Analysis of Synthetic Peptides

### Materials and Methods

### Peptide Synthesis

Peptide synthesis and purification was performed. Peptides were synthesized by standard Fmoc solid-phase synthesis using 2-Cl-Trt resin [GB Fields, R. Hammami]. Briefly, the Fmoc protecting group was removed from the resin by two 10 min treatments with 20% piperidine in dimethylformamide (DMF, v/v) and amino acid coupling was performed with Fmoc-XaaOH (3 equivalents), 2-(6-Chloro-1H-benzotriazole-1-yl)-1,1,3,3-tetramethylaminium hexafluorophosphate (HCTU, 3 equivalents) and N-methylmorpholine (12 equivalents) in dimethylformamide (DMF, 2×30 min). The synthesized peptides were released by treating the resin with 20% hexafluoro-2-propanol (HFIP) in dichloromethane (DCM) for 30 min [206]. Side chain deprotection was achieved by treating the peptides with TFA/Triisopropylsilane (TIPS)/H2O (95:2.5:2.5, v/v/v) for 3 h. The resulting peptides were precipitated with cold ether and purified by RP-HPLC with a Shimadzu Prominence instrument (Columbia, Md., USA) on a Vydac 218 MS column (22.0×250 mm, 300 Å, 10 μm, C18) using 0.1% TFA/H2O (solvent A) and 0.1% TFA/CH3CN (solvent B) with a linear gradient of 10-100% solvent B for 20 min at 10 mL/min and UV detection at 220 nm and 254 nm. After freeze-drying, the purified peptides were characterized by matrix-assisted laser desorption-ionization time-of-flight mass spectrometry (MALDI-TOF) on an AB SCIEX 4800 Plus MALDI-TOF/TOF instrument using alpha-cyano-4-hydroxycinnamic acid as matrix.

### Glucose Uptake Experiments

Glucose uptake experiments were conducted as described by Roblet 2013. L6 skeletal muscle cells were grown in an α-minimum essential medium (α-MEM) containing 2% (v/v) fetal bovine serum (GBS) in an atmosphere of 5% CO2 at 37° C. [Tremblay 2001]. Cells were plated at 600,000 cells/plate in 24-well plates to obtain about 25,000 cells/mL. The cells were incubated 7 days, to reach their complete differentiation to myotubes (7 days post-plating). L6 myotubes were deprived of GBS for 3 h, with a α-MEM containing 0% of GBS. Then, the cells were incubated for 75 minutes, with 10 μl of EDUF fractions at a concentration of 1 μg/mL and 1 ng/mL. Finally, insulin was added (10 μl at 1.10-5M) for 45 min. Experiments were repeated 9 times, and each repetition was run in triplicate. After experimental treatments, cells were rinsed once with 37° C. HEPES-buffered solution (20 mM HEPES, pH 7.4, 140 mM NaCl, 5 mM KCl, 2.5 mM MgSO4, and 1 mM CaCl₂)) and were subsequently incubated in HEPES-buffered solution containing 10 μM 2-deoxyglucose and 0.3 μCi/mL 2-deoxy-[3H] glucose for 8 minutes. Then, the cells were rinsed three times with 0.9% NaCl solution at 4° C. and then frozen. The next day, the cells were disrupted by adding 500 μl of a 50 mM NaOH solution. The radioactivity was determined by scintillation.

### Hepatic Glucose Production Experiments

Hepatic glucose production experiments were conducted as described by Chevrier et al, (2015). Briefly, FAO rat hepatocytes were grown and maintained in monolayer culture in Roswell Park Memorial Institute medium (RPMI) containing 10% FBS in an atmosphere of 5% CO₂ at 37° C. Cells were plated at 4.10⁶ cells/plate. FAO cells were deprived with 1 mL/well of RPMI without FBS, and the EDUF's fractions were added at 10 μl/well with or without insulin at 1 nmol. Cells were washed three times with PBS, then incubated for 5 h (in an atmosphere of 5% CO₂ at 37° C.) with the peptide fractions in the presence or absence of insulin at 1 nmol in a hepatic glucose production medium (glucose-free DMEM containing sodium bicarbonate at 3.7 g/L, 2 mmol sodium pyruvate, and 20 mmol sodium L-lactate. Glucose production was measured in the medium by using the Amplex Red Glucose/Glucose Oxidase Assay kit (Invitrogen). Results shown are the mean response of at least 6 independent experiments realized in triplicate.

### Results

### Synthetic Peptide Regulation of Glucose Uptake

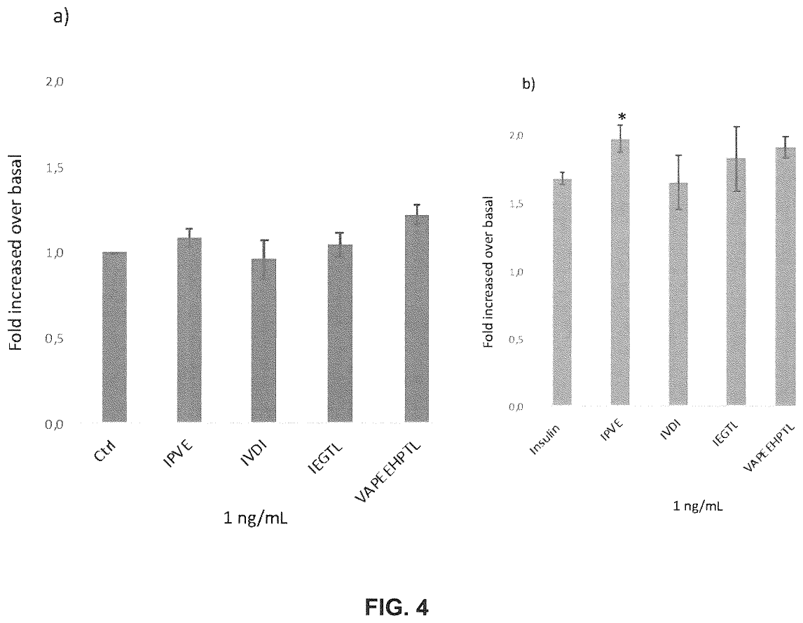

IPVE was synthetized and its capacity to increase the glucose uptake was tested in basal and insulin stimulation conditions (FIG. 4). In the presence of insulin, IPVE demonstrated a significant enhancement of the glucose uptake (17%) compared to the insulin control (P=0.016).

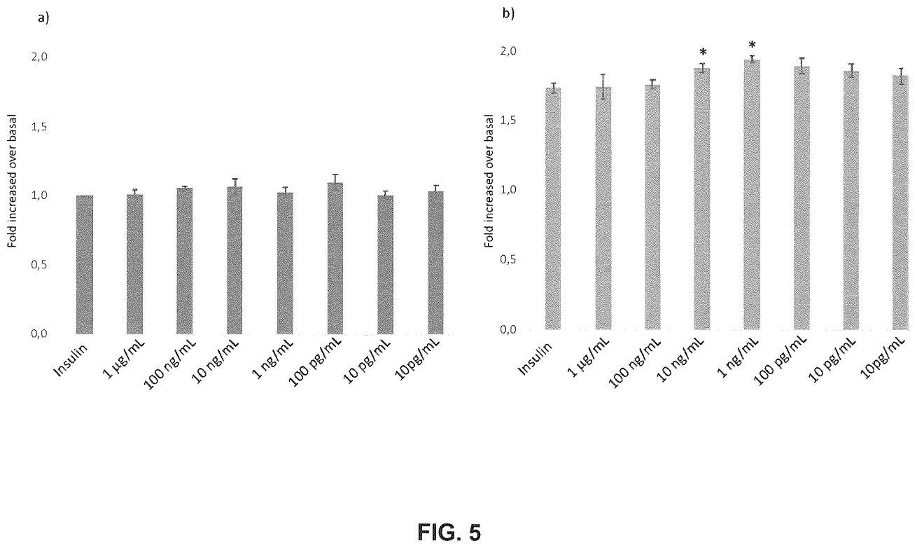

Next, the dose-response effect to IPVE was tested (FIG. 5) with concentrations from 1 μg/mL to 10 pg/mL. These results confirmed the capacity to IPVE to improve the glucose uptake in muscle cells, as it was able to increase the bioactivity at 10 ng/mL and 1 ng/mL. Also, this result demonstrated that the response of IPVE was dose-dependent, since for the highest (>10 ng/mL) and the lowest (<100 pg/mL) concentrations tested, IPVE presented no effect on the glucose uptake.

### Synthetic Peptide Regulation of Hepatic Glucose Production

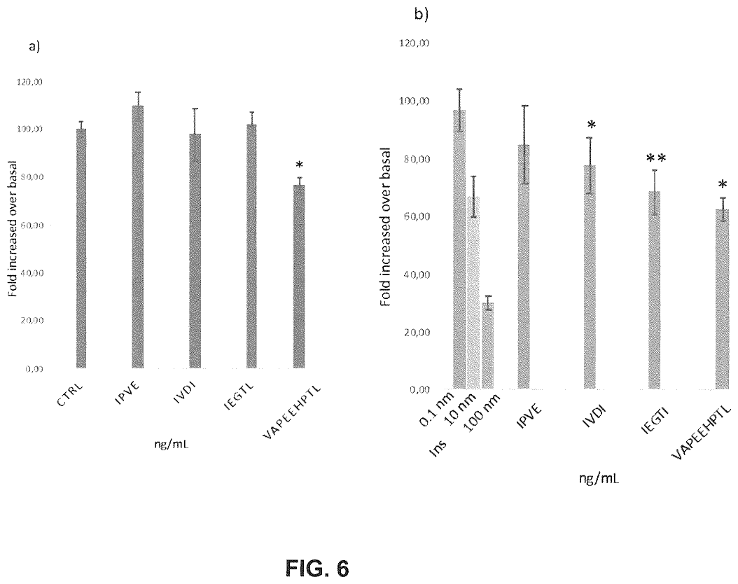

The capacity of IVDI, IEGTL, and VAPEEHPTL to regulate the hepatic glucose production (“HGP”) was investigated and results are presented in FIG. 6. The three peptides were tested in 6 repetitions in basal and insulin conditions. For the insulin condition, the peptides were incubated with insulin at 0.1 nm, and the statistical comparisons were performed between the insulin control at 1 nm and the peptides incubated with insulin at 0.1 nm. IVDI and IEGTL demonstrated a decrease of the HGP by 20% and 30% respectively when compared to insulin at 0.1 nm. Moreover, IEGTL incubated with insulin at 0.1 nm showed the same capacity to decrease the HGP than insulin alone at 10 nm. VAPEEHPTL demonstrated a 20% decrease in HGP in the basal condition and an 18% decrease in the insulin-stimulated condition.

### Regulating Blood Glucose in Humans:

The effect of the peptides, compositions, and combinations described herein on glucose regulation is shown in human patients with type-2 diabetes. Patients are divided into two groups: treatment and control. Both patient groups are administered the same type and quantity of food, wherein the food has a glycemic index value of 56 or higher. After the food is administered, patients in the treatment group are given a composition comprising one or more peptides of the amino acid sequence of any one of SEQ ID NOs: 1-4, while patients in the control group are given a placebo composition that does not have an effect on glucose uptake, production, or regulation. Following administration of the placebo or the treatment composition, the blood glucose levels of the patients in both groups is measured. On average, patients in the treatment group are observed have a lower blood glucose level than the patients in the control group.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety.

TABLE 1
Identified peptides

| Peptide | | SEQ ID |
|---|---|---|
| Sequence | Bioactivity | NO: |
| IPVE | Increases glucose uptake | 1 |
| IEGTL | Decreases hepatic glucose production | 2 |
| IVDI | Decreases hepatic glucose production | 3 |
| VAPEEHPTL | Decreases hepatic glucose production | 4 |

TABLE 2
Characterization of glucoregulatory peptides

| Molecular weight | Retention time | Potential | Net | |
|---|---|---|---|---|
| (Avg) (Da) | (Avg) (min) | sequence | charge | pI |
| 991.4967 | 15.786 | VAPEEHPTL | — | 4.50 |
| 531.2903 | 17.26 | IEGTL | — | 4.00 |
| 458.2737 | 22.331 | IVDI | — | 3.80 |
| 456.2581 | 10.165 | IPVE | — | 4.60 |

^{a} calculated at pH 6.00

### REFERENCES

Aider, M., Arul, J., Mateescu, M. A., Brunet, S., & Bazinet, L. (2006). Electromigration behavior of a mixture of chitosan oligomers at different concentrations. Journal of Agricultural and Food Chemistry, 54(26), 10170-10176. http://doi.org/10.1021/jf061653n.
Aider, M., Brunet, S., & Bazinet, L. (2009). Effect of solution flow velocity and electric field strength on chitosan oligomer electromigration kinetics and their separation in an electrodialysis with ultrafiltration membrane (EDUF) system. Separation and Purification Technology, 69(1), 63-70. http://doi.org/10.1016/j.seppur.2009.06.020
Bazinet, L., Doyen, A., & Roblet, C. (2010). Electrodialytic phenomena, associated electromembrane technologies and applications in the food, beverage and nutraceutical industries.
Bedard, S., Marcotte, B., & Marette, A. (1997). Cytokines modulate glucose transport in skeletal muscle by inducing the expression of inducible nitric oxide synthase. The Biochemical Journal, 325 (Pt 2, 487-493. http://doi.org/10.1016/S0021-9150(96)05976-X.
Chevrier, G., Mitchell, P. L., Rioux, L.-E., Hasan, F., Jin, T., Roblet, C. R., Marette, A. (2015). Low-Molecular-Weight Peptides from Salmon Protein Prevent Obesity-Linked Glucose Intolerance, Inflammation, and Dyslipidemia in LDLR−/−/ApoB100/100 Mice. The Journal of Nutrition, 145(7), 1415-22. http://doi.org/10.3945/jn.114.208215.
Drotningsvik, A., S. A. Mjøs, D. M. Pampanin, R. Slizyte, A. Carvajal, T. Remman, I. Høgøy, O. A. Gudbrandsen, Dietary fish protein hydrolysates containing bioactive motifs affect serum and adipose tissue fatty acid compositions, serum lipids, postprandial glucose regulation and growth in obese Zucker fa/fa rats, Br. J. Nutr. 116 (2016) 1336-1345. doi:10.1017/s0007114516003548.
Fields, G B, R. L. NOBLE, Solid phase peptide synthesis utilizing 9-fluorenylmethoxycarbonyl amino acids, Int. J. Pept. Protein Res. 35 (1990) 161-214. doi:10.1111/j.1399-3011.1990.tb00939.x.
Hammami, R., A. Gomaa, E. Biron, I. Fliss, M. Subirade, F. Bédard, Lasso-inspired peptides with distinct antibacterial mechanisms, Amino Acids. 47 (2015) 417-428. doi:10.1007/s00726-014-1877-x.
Harkness T A, Shea K A, Legrand C, Brahmania M, Davies G F. (2004). A functional analysis reveals dependence on the anaphase-promoting complex for prolonged life span in yeast. Genetics 168:759-74.
Henaux L, J Thibodeau, G Pilon, T Gil, A Marette, and L Bazinet. How Charge and Triple Size-Selective Membrane Separation of Peptides from Salmon Protein Hydrolysate Orientate their Biological Response on Glucose Uptake. Int. J. Mol. Sci. 2019: 20(8), 1939.
International Diabetes Federation, Atlas du Diabete de la FID, 2017.
Lavigne, C., A. Marette, H. Jacques, Cod and soy proteins compared with casein improve glucose tolerance and insulin sensitivity in rats., Am. J. Physiol. Endocrinol. Metab. 278 (2000) E491-E500.
Lavigne, C., F. Tremblay, G. Asselin, H. Jacques, A. Marette, Prevention of skeletal muscle insulin resistance by dietary cod protein in high fat-fed rats., Am. J. Physiol. Endocrinol. Metab. 281 (2001) E62-E71.
Liaset, B., L. Madsen, Q. Hao, G. Criales, G. Mellgren, H. Marschall, P. Hallenborg, M. Espe, L. Frøyland, K. Kristiansen, Fish protein hydrolysate elevates plasma bile acids and reduces visceral adipose tissue mass in rats, BBA—Mol. Cell Biol. Lipids. 1791 (2009) 254-262. doi:10.1016/j.bbalip.2009.01.016.
Ouellet, V., J. Marois, S. J. Weisnagel, H. Jacques, Dietary cod protein improves insulin sensitivity in insulin-resistant men and women: a randomized controlled trial, Diabetes Care. 30 (2007) 2816-2821. doi:10.2337/dc07-0273.
Ouellet, V., S. J. Weisnagel, J. Marois, J. Bergeron, P. Julien, R. Gougeon, A. Tchernof, B. J. Holub, H. Jacques, Dietary Cod Protein Reduces Plasma C-Reactive Protein in Insulin-Resistant Men and Women 1-3, J. Nutr. 2008 (2008) 2386-239. doi: 10.3945/jn.108.092346.studies.
Parolini, C., R. Vik, M. Busnelli, B. Bjørndal, S. Holm, T. Brattelid, S. Manzini, G. S. Ganzetti, F. Dellera, B. Halvorsen, P. Aukrust, C. R. Sirtori, J. E. Nordrehaug, J. Skorve, R. K. Berge, G. Chiesa, A salmon protein hydrolysate exerts lipid-independent anti-atherosclerotic activity in apoE-deficient mice, PLoS One. 9 (2014) 1-9. doi:10.1371/journal.pone.0097598.
Pilon, G., J. Ruzzin, L. E. Rioux, C. Lavigne, P. J. White, L. Froyland, H. Jacques, P. Bryl, L. Beaulieu, A. Marette, Differential effects of various fish proteins in altering body weight, adiposity, inflammatory status, and insulin sensitivity in high-fat-fed rats, Metabolism. 60 (2011) 1122-1130. doi:10.1016/j.metabol.2010.12.005.
Roblet, C., Akhtar, M. J., Mikhaylin, S., Pilon, G., Gill, T., Marette, A., & Bazinet, L. (2016). Enhancement of glucose uptake in muscular cell by peptide fractions separated by electrodialysis with filtration membrane from salmon frame protein hydrolysate. Journal of Functional Foods, 22, 337-346. http://doi.org/10.1016/j.jff.2016.01.003.
Roblet, C., Doyen, A., Amiot, J., & Bazinet, L. (2013). Impact of pH on ultrafiltration membrane selectivity during electrodialysis with ultrafiltration membrane (EDUF) purification of soy peptides from a complex matrix. Journal of Membrane Science, 435, 207-217. http://doi.org/10.1016/j.memsci.2013.01.045.
Stumvoll, M., B. J. Goldstein, T. W. van Haeften, Type 2 diabetes: principles of pathogenesis and therapy., Lancet (London, England). 365 (2005) 1333-46. doi: 10.1016/S0140-6736(05)61032-X.
Suwal, S., Roblet, C., Amiot, J., & Bazinet, L. (2015). Presence of free amino acids in protein hydrolysate during electroseparation of peptides: Impact on system efficiency and membrane physicochemical properties. Separation and Purification Technology, 147, 227-236. http://doi.org/10.1016/j.seppur.2015.04.014.
Tremblay, F., & Marette, A. (2001). Amino acid and insulin signaling via the mTOR/p70 S6 kinase pathway: A negative feedback mechanism leading to insulin resistance in skeletal muscle cells. The Journal of Biological Chemistry, 276(41), 38052-38060. http://doi.org/10.1074/jbc.M106703200.
Tremblay, F., C. Lavigne, H. Jacques, A. Marette, Dietary Cod Protein Restores Insulin-Induced Activation of Phosphatidylinositol 3-Kinase/Akt and GLUT4 Translocation to the T-Tubules in Skeletal Muscle of High-Fat-Fed Obese Rats, Diabetes Care. 52 (2003) 29-37.
Wergedahl, H., B. Liaset, O. A. Gudbrandsen, E. Lied, M. Espe, Z. Muna, S. Mørk, R. K. Berge, Fish protein hydrolysate reduces plasma total cholesterol, increases the proportion of HDL cholesterol, and lowers acyl-CoA:cholesterol acyltransferase activity in liver of Zucker rats., J. Nutr. 134 (2004) 1320-1327.
Wild, S., G. Roglic, A. Green, R. Sicree, H. King, Estimates for the year 2000 and projections for 2030, Diabetes Care. 27 (2004) 1047-1053. doi:10.2337/diacare.27.5.1047 Diabetes Care May 2004 vol. 27 no. 5 1047-1053.
Zhu, C F., H. B. Peng, G. Q. Liu, F. Zhang, Y. Li, Beneficial effects of oligopeptides from marine salmon skin in a rat model of type 2 diabetes, Nutrition. 26 (2010) 1014-1020. doi:10.1016/j.nut.2010.01.011.

1. A peptide comprising (i) an amino acid sequence as shown in SEQ ID NO: 1 (IPVE); or (ii) a peptide comprising at least 75% sequence identity with the amino acid sequence as shown in SEQ ID NO: 1 that increases glucose uptake.

2. The peptide of claim 1, comprising or consisting of the amino acid sequence shown in SEQ ID NO: 1.

3. (canceled)

4. The peptide of claim 1, wherein the peptide is less than 50, 45, 40, 35, 30, 25, 20, 15, or 10 amino acids in length.

5. The peptide of claim 1, wherein the peptide is modified for cell permeability, stability or bioavailability.

6. A composition comprising the peptide of claim 1 and a carrier.

7. A peptide comprising (i) an amino acid sequence as shown in any one of SEQ ID NO: 2 (IEGTL), SEQ ID NO: 3 (IVDI) or SEQ ID NO: 4 (VAPEEHPTL); or (ii) a peptide comprising at least 67, 75, 80, or 90% sequence identity with the amino acid sequence as shown in any one of SEQ ID NOs: 2-4 that decreases hepatic glucose production.

8. The peptide of claim 7, comprising or consisting of the amino acid sequence of any one of SEQ ID NOs: 2-4.

9. (canceled)

10. The peptide of claim 7, wherein the peptide is less than 50, 45, 40, 35, 30, 25, 20, 15 or 10 amino acids in length.

11. The peptide of claim 7, wherein the peptide is modified for cell permeability, stability or bioavailability.

12. A composition comprising the peptide of claim 7 and a carrier.

13. A composition comprising at least two peptides of claim 1, optionally at least two, at least three or four peptides of SEQ ID NOs: 1-4 and a carrier.

14. A method of increasing glucose uptake in a subject in need thereof, the method comprising administering to the subject the peptide of claim 1.

15. (canceled)

16. (canceled)

17. A method of decreasing hepatic glucose production in a subject in need thereof, the method comprising administering to the subject the peptide of claim 7.

18. (canceled)

19. (canceled)

20. A method of regulating glucose levels in a subject in need thereof, the method comprising administering to the subject the peptide of claim 1.

21. (canceled)

22. (canceled)

23. A method of treating diabetes, optionally type 1 or type 2 diabetes, in a subject in need thereof, the method comprising administering to the subject the peptide of claim 1.

24. (canceled)

25. (canceled)

26. A method of treating metabolic syndrome (MS) by reducing hyperglycemia in a subject in need thereof, the method comprising administering to the subject the peptide of claim 1.

27. (canceled)

28. (canceled)

29. The method of claim 14, wherein the subject is a diabetic subject.

30. The method of claim 14, wherein the subject is a mammal, optionally a dog cat, horse, or human.

31. The method of claim 14, wherein the peptide is administered orally or intravenously.

32. (canceled)

33. A method of obtaining the peptide of claim 1, the method comprising:
providing a homogenized salmon frame or fraction;
precipitating proteins from the homogenized fraction;
hydrolyzing the precipitated proteins to form a hydrolyzed solution;
filtering the hydrolyzed solution using an ultrafiltration membrane to generate a filtrate; and
isolating the peptide from the filtrate, optionally isolating peptides of SEQ ID NO: 1, SEQ ID NO: 2, SEQ ID NO: 3, and SEQ ID NO: 4 into separate fractions.
